(19) United States
(12) Patent Application Publication
(10) Pub. No.: US 2007/0141176 A1
(43) Pub. Date: Jun. 21, 2007

(54) Elemental sulfur as an oral or parentral medical product

(76) Inventor: Abdalla Magd Ahmed Kotb, Cairo (EG)

Correspondence Address: Jay S Cinamon;Abelman Frayne and Schwab, 666 Third Avenue, New York, NY, 10017-5621, US

(21) Appl. No.: 10/584,697

(22) PCT Filed: Feb. 16, 2004
(86) PCT No.: PCT/EG04/00004

§ 371 Date: Jun. 22, 2006

(30) Foreign Application Priority Data
Dec. 23, 2003 (EG) 2003121103

(51) Int. Cl. A61K 33/04

(52) U.S. Cl. 424/705

(57) ABSTRACT

Title element and its acid addition salts and derivatives are physiologically acceptable, essential and are readily converted to acceptable counter parts by established procedures, they are pharmacologically active on the liver, lungs, hematopoetic system and all body systems and are thus useful when administered to warm blooded animals to induce detoxification of many endogenous and exogenous metabolites. They are useful in terminating diseases associated with Glutathione S Transferase and Epoxide Hydrolase disorders. These compounds are prepared as elemental or as salts or as acid addition salts and derivatives compounds. They are simply elemental or compounded into different dosage—multi form medicament compositions.

### THE PRIOR ART

Environmental pollutants as Epoxides, arene oxide, nitro groups, hydroxylamines and aflatoxins need Glutathione conjugation where the endogenous reactant is Glutathione in the presence of Glutathione S Transferase (cytosol microsomes). Since Glutathione is the major intracellular soluble sulfahydryl—containing compound, factors that regulate the biosynthesis and the composition have important consequences. The Glutathione conjugation is a common pathway for detoxification of primary metabolites to reduced Glutathione. These water soluble secondary metabolites are readily excreted in the bile and urine. Glutathione conjugation defects leads to accumulation of environmental pollutants. Treatment of the resultant diseases and syndromes were targeted at supplying sulfur as sulphates of different organic and inorganic substances with side effects related to these substances.

### THE PROBLEM

All the earlier experiences addressed the symptomatology of the disease and none addressed the aetiology which was obscure. None provided the sulfur to bypass the deficiency of the glutathione S transferase to detoxify all the ingested or inhaled toxins.

### THE NEW IN THE INVENTION

Elemental Sulfur and its acid addition salts and derivatives for Glutathione S Transferase heterozygous and homozygous disorders and Epoxide Hydrolase heterozygous and homozygous disorders.

Title element and its acid addition salts and derivatives are physiologically acceptable, essential and are readily converted to acceptable counter parts by established procedures, they are pharmacologically active on the liver, lungs, hematopoetic system and all body systems and all malignancies and the autoimmune and immune mediate disorders and are thus useful when administered to warm blooded animals to induce detoxification of many endogenous and exogenous metabolites. They are useful in terminating diseases associated with Glutathione S Transferase and Epoxide Hydrolase disorders.

The elemental sulfur provides the body and liver cells by the essential sulfur to bind to toxic material and thus is eliminated in bile. Sulfur is absorbed from the small intestines as sulfates with essential amino-acids or as a salt to other acids.

These compounds are prepared as elemental or as salts or as acid to addition salts and derivatives compounds. They are simply elemental or compounded into different dosage—multi form medicament compositions.

### THE DETAILED DESCRIPTION

Pure sulfur element and/or its acid addition salts and derivatives are prepared by all industrial techniques and processes, and bound by any binder.

### INDUSTRIAL APPLICABILITY

Inclusion of Elemental Sulfur and/or its acid addition salts and derivatives in management protocols of Glutathione S Transferase heterozygous and homozygous disorders and Epoxide Hydrolase heterozygous and homozygous disorders, and glutathione depletion associated syndromes.

1. I claim the patent as this is an invention and industrially applicable, involving new application of known industrial processing. (Part 1-Article 1).

2. I claim the right that the patent shall belong to the inventor or his successor in title.

3. I claim the protection period of 20 years. (Part 1—Article 9).

4. I claim the right to end product protection.

5. I claim the right to prevent a third party from exploiting the invention by any means: importing, using, selling, advertising or distributing the product (Part 1—Article 10).

6. I claim the right for manufacturing, processing sulfur in its elemental form or in acid salts, combined and/or formulated with R as a medical product in all forms as medicine, medical foods, artificial milks and all medicaments.
R degree is, independently, any member selected from the group of any acids and any acid salts derivatives.
R is —O, where the sulfate formed can be combined with any acids and any acid salt derivatives.
R is all compounds able to form final formulation releasing elemental sulfur (subject invention).
